(19) United States
(12) Patent Application Publication
(10) Pub. No.: US 2003/0045695 A1
(43) Pub. Date: Mar. 6, 2003

(54) Ribozyme for detecting gene sequence

(76) Inventors: Yasuo Komatsu, Kanagawa (JP); Eiko Ohtsuka, Kanagawa (JP)

Correspondence Address: Pillsbury Winthrop LLP, Suite 200, 11975 El Camino Real, San Diego, CA, 92130, US

(21) Appl. No.: 10/032,794

(22) Filed: Dec. 27, 2001

(30) Foreign Application Priority Data
Dec. 27, 2000 (JP) 399155/2000

(51) Int. Cl. C07H 21/02; C12Q 1/68

(52) U.S. Cl. 536/23.1; 435/6

(57) ABSTRACT

A hairpin ribozyme activated by changes in stem-and-loop three-dimensional structure due to hybridization with an oligonucleotide. Presence or absence of a target nucleotide sequence in a sample can be detected and ribozyme activity can be regulated.

### PRIORITY INFORMATION

This application claims priority to Japanese Application Serial No. 399155/2000, filed Dec. 27, 2000.

### FIELD OF THE INVENTION

The present invention relates to a hairpin ribozyme activated by changes in its stem-and-loop three-dimensional structure due to hybridization with an oligonucleotide, DNA encoding ribonucleotides constituting said ribozyme, a recombinant vector comprising said DNA, a host cell into which the recombinant vector has been introduced, and a method of detecting a target nucleotide sequence with the above ribozyme.

### BACKGROUND OF THE INVENTION

It has been found that plus strand of satellite RNA of tobacco ringspot virus and plus strand RNA and minus strand RNA of avocado sandwich viroid are cleaved by their own catalytic activity in the presence of Mg²⁺ (see Prody, G. A. et al., (1986) Science 231, 1577-1580 and Hutchins, C. J. et al., (1986) Nucleic Acids Res. 14, 3627-3640). The nucleotide sequences in the vicinity of the cleavage sites of these RNAs have common features, and the two-dimensional structures of these RNAs have been predicted from such common features. Uhlenbeck engineered a 19-mer short-strand RNA fragment based on these common sequences, and showed that this fragment catalytically cleaves a 24-mer RNA (Uhlenbeck, O. C. (1987) Nature 328, 596-600).

In addition to satellite RNA of a viroid or virus, it is reported that a transcript of satellite DNA from a newt also had the nucleotide sequence of this ribozyme (Epstein, L. M. and Gall, J. G. (1987) Cell, 48, 535-543). It has been found that if 2 types of 21-mer RNA having nucleotide sequences in the vicinity of a cleavage site of this transcript of satellite DNA from newt are chemically synthesized, and one RNA is then added to other RNA, a cleavage reaction will occur at an identical site with natural reaction (Koizumi, M. et al., (1988) FEBS Lett. 228, 228-230). Furthermore, a method that cleaves another RNA or a polyribonucleotide molecule using a ribozyme has been developed based on this result (Koizumi, M. et al., (1989) Nucleic Acids Res. 17, 7059-7071).

Meanwhile, it has been clarified that the minus strand of satellite RNA of tobacco ringspot virus also effects a cleavage reaction, and that cleavage is effected at a specific site (Buzayan, J. M. et al., (1986) Nature 323, 349-353). Also, the minimum region of RNA essential for this cleavage has been clearly identified (Hampel, A. and Tritz, R. (1989) Biochemistry, 28, 4929-4933). RNA having this catalytic activity comprises 50 nucleotides, and a model having a hairpin loop structure inside this RNA has been proposed and given the name “hairpin ribozyme.” By substituting a nucleotide of this hairpin ribozyme into a different nucleotide, several nucleotides that are important in a cleavage reaction have been clearly identified (Chowrira, B. M. et al., (1991) Nature 354, 320-322, and Sekiguchi, A. et al. (1991) Nucleic Acids Res. 19, 6833-6838). Also, Burke's group have clearly identified an important nucleotide sequence in the ligation and cleavage reaction of a hairpin ribozyme by means of a DNA having a random nucleotide sequence and an in vitro selection method using the PCR (polymerase chain reaction) method (Berzal-Herranz, A. et al., (1992) Gene & Development 6, 129-134). Further, it has also been clarified that a catalytic reaction proceeds even in an RNA that lacks a hairpin loop area (Sekiguchi, A. et al., (1991) Nucleic Acids Res. 19, 6833-6838). In addition, Koizumi et al. found that transforming the hairpin loop of a hairpin ribozyme into a thermodynamically stabilized sequence, results in a ribozyme having a higher activity than the normal type (Japanese Patent Application Laying-Open (kokai) No. 6-181758).

Komatsu et al. synthesized RNAs in which a hydroxyl group at the 5′ terminus of a substrate is bound to a hydroxyl group at the 3′ terminus of the shorter strand of a double-strand hairpin ribozyme via a propanediol phosphate linker that has repeated one to 13 times. Inspection of cleavage activity of these RNAs revealed that, those, which bound a substrate to a chain of a ribozyme via a linker having a repetition of propanediol phosphate seven times or more, had greater activity than the wild type. This indicates that there exists a structure that is bent between the second helix and the third helix among the four helices of hairpin ribozymes [Komatsu, Y et al., (1994) J. Am. Chem. Soc. 116, 3602]. Ohtsuka et al. had attempted to convert the binding site in the hairpin ribozyme domain while retaining the higher order structure of bent hairpin ribozyme shown herein (catalytic activity domain and substrate-binding domain are linked at a site of helix 1 and helix 4 via polymeric polyribonucleotides). As a result, they have found that the polyribonucleotide has still higher ribozyme activity even with the conversion of binding site in the domain [Japanese Patent Application Laying-Open (kokai) No. 8-131163]. Furthermore, Komatsu et al. have constructed a ribozyme comprising 3 domains in which a hairpin ribozyme, originally comprising two domains, a catalytic activity domain and a substrate-binding domain, is bound to another cleavage domain, and found that the ribozyme has a self-trimming activity for self-cleaving during transcription [Japanese Patent Application Laying-Open (kokai) No. 10-215876].

On the other hand, an attempt has been made to cleave RNA associated with diseases by artificially expressing a ribozyme in a cell [for example, Bratty, J. et al., (1993) Biochim. Biophys. Acta 1216, 345-359]. In that case, a method may be adopted where a ribozyme is administered as DNA encoding the ribozyme or where a retrovirus vector is used. In either method, a system is employed wherein ribozyme genes are expressed under the control of a mammalian promoter.

Further, we have already reported that a hammerhead ribozyme can be activated by binding with an externally-added oligonucleotide and forming a pseudo-half-knot structure [Komatsu et al., J. Mol. Biol., 2000].

Under the above circumstances, we have made various studies for the purpose of converting the hairpin ribozyme (FIG. 1), which is a type of ribozyme being different from a hammerhead ribozyme, so as to be activated by addition of an oligonucleotide.

A wild-type hairpin ribozyme is constituted by two domains as shown in FIG. 1 in which RNA (ribozyme) is cleaved at the site indicated with an arrow. Domain I comprises helix 1, helix 2, and internal loop A. Domain II comprises helix 3, helix 4, and internal loop B. Formation of base pairs is important for the 4 helices although conversion to other nucleotides is possible. In contrast, two internal loops substantially consist of nucleotides that are essential to cleavage activity. According to the technique described in Japanese Patent Laying Open (kokai) No. 6-181758, a hairpin loop, corresponding to helix 4 of the hairpin ribozyme, is thermodynamically stabilized, and ribozyme activity is not regulated by an externally-added nucleic acid. The ribozyme is constantly in an active state. Thus, this technique cannot be utilized for detecting genes. The technique described in Japanese Patent Laying Open (kokai) No.10-215876 also has a helix 4 structure, and thus, constantly has an active conformation instead of a conformation in which activity can be regulated.

The object of the present invention is to provide a ribozyme, which dose not effect self-cleavage reaction by itself, but, by binding between an oligonucleotide having a subject nucleotide sequence and the ribozyme, the ribozyme changes from an inactive conformation to an active conformation, and exhibits RNA cleaving activity, and a method for cleaving a polyribonucleotide using the ribozyme.

### SUMMARY OF THE INVENTION

The present invention provides a polyribonucleotide, which exhibits cleavage activity (hereinafter referred to as a “ribozyme”) only in the presence of an oligonucleotide, and a method for cleaving a substrate polyribonucleotide using the polyribonucleotide (ribozyme).

We have found that ribozyme activity can be regulated with a polyribonucleotide by converting a hairpin loop so as to activate the ribozyme through binding with an externally-added oligonucleotide, although the hairpin ribozyme is inactive by itself. This had led to the completion of the present invention. FIG. 2 shows an embodiment of ribozyme activation, i.e., an inactive conformation before binding with an oligonucleotide and an active conformation after binding with an oligonucleotide.

The present invention provides the following (1) to (21).

(1) A hairpin ribozyme activated by changes in its stem-and-loop three-dimensional structure due to hybridization with an oligonucleotide.

(2) The hairpin ribozyme according to (1) above, wherein the hybridization with an oligonucleotide is constituted by 3 to 23 base pairs.

(3) The hairpin ribozyme according to (1) or (2) above, wherein the oligonucleotide is a part of a target nucleotide sequence.

(4) The hairpin ribozyme according to any one of (1) to (3) above, which is a cis-form ribozyme which first self-cleaves upon activation.

(5) The hairpin ribozyme according to (4) above, which exhibits a complex structure with an oligonucleotide as shown in general formula (I) or (II)

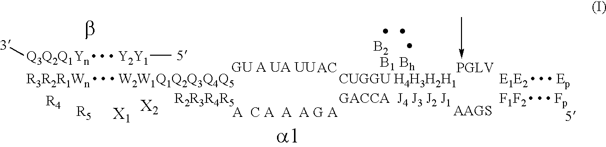

[wherein α1 represents a ribozyme sequence and β represents an oligonucleotide sequence:

in which U represents a uracil nucleotide, C represents a cytosine nucleotide, A represents an adenine nucleotide, and G represents a guanine nucleotide;

B1 to Bh, E1 to Ep, H1 to H4, Q1 to Q5, W1 to Wn, and X1 and X2, which may be the same or different, each represent any of a uracil nucleotide, an adenine nucleotide, a cytosine nucleotide, or a guanine nucleotide;

F1 to Fp, J1 to J4, R1 to R5, and Y1 to Yn each represent a nucleotide, which is complementary to E1 to Ep, H1 to H4, Q1 to Q5, and W1 to Wn, respectively;

S represents an adenine nucleotide or a cytosine nucleotide;

P represents any of a uracil nucleotide, an adenine nucleotide, a cytosine nucleotide, or a guanine nucleotide;

L represents any of a uracil nucleotide, an adenine nucleotide, or a cytosine nucleotide;

V represents an adenine nucleotide when S is a cytosine nucleotide and represents a uracil nucleotide or a cytosine nucleotide when S is an adenine nucleotide; and

h is an integer from 3 to 20, n is an integer from 1 to 10, and p is an integer from 1 to 10.]

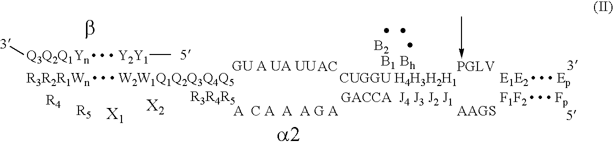

[wherein α2 represents a ribozyme sequence and β represents an oligonucleotide sequence with symbols used in this formula conveying the same meanings as used in general formula (I).]

(6) The hairpin ribozyme according to (1) or (2) above, which is a trans-form ribozyme which cleaves another ribonucleotide sequence through activation.

(7) The hairpin ribozyme according to (6) above, which exhibits a complex structure with an oligonucleotide as shown in general formula (III)

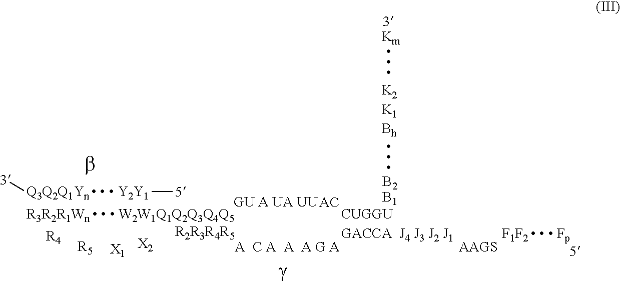

[wherein γ represents a ribozyme sequence and β represents an oligonucleotide sequence:

in which U represents a uracil nucleotide, C represents a cytosine nucleotide, A represents an adenine nucleotide, and G represents a guanine nucleotide;

B1 to Bh, F1 to Fp, J1 to J4, K1 to Km, Q1 to Q5, W1 to Wn, and X1 and X2, which may be the same or different, each represent any of a uracil nucleotide, an adenine nucleotide, a cytosine nucleotide, or a guanine nucleotide;

R1 to R5 and Y1 to Yn each represent a nucleotide, which is complementary to Q1 to Q5 and W1 to Wn, respectively;

S represents an adenine nucleotide or a cytosine nucleotide; and

h is an integer from 3 to 20, m is an integer from 1 to 10, n is an integer from 1 to 10, and p is an integer from 1 to 10.]

(8) An isolated DNA encoding a ribonucleotide which constitutes the hairpin ribozyme according to any one of (1) to (7) above.

(9) A recombinant vector comprising the DNA according to (8) above.

(10) A host cell into which the recombinant vector according to (9) above has been introduced.

(11) A method for activating a hairpin ribozyme, which comprises changing a stem-and-loop three-dimensional structure by hybridization between an oligonucleotide and an inactive ribozyme.

(12) The method for activating a hairpin ribozyme according to (11) above, wherein one or more nucleotides in the oligonucleotide are 2′-O-methylated.

(13) A method for detecting a target nucleotide sequence with the hairpin ribozyme according to any one of (1) to (7) above.

(14) The method for detecting according to (13) above, which comprises detecting the presence of a target nucleotide sequence in a sample held on a DNA chip.

(15) A method for detecting a target nucleotide sequence, which comprises detecting a fragment created by self-cleavage of the hairpin ribozyme according to any one of (1) to (5) above.

(16) The method for detecting according to any one of (13) to (15) above, which comprises detecting a cleavage fragment using a fluorochrome or a radioactive label.

(17) A detection kit for a target nucleotide sequence in a sample, comprising the hairpin ribozyme according to any one of (1) to (7) above.

(18) A method for cleaving a ribonucleotide sequence, using the hairpin ribozyme according to any one of (1) to (7) above.

(19) The method for cleaving according to (18) above, wherein administration of the hairpin ribozyme according to any one of (1) to (7) above is carried out separately from that of the oligonucleotide.

(20) The method for cleaving according to (17) or (18) above, wherein one or more nucleotides in the oligonucleotide are 2′-O-methylated.

(21) A pharmaceutical composition comprising the hairpin ribozyme according to any one of (1) to (7) above.

According to the present invention, engineering of α1 chain or α2 chain in a sequence represented by general formula (I) or (II) based on the β chain sequence in general formula (I) or (II) enables engineering of a ribozyme that effects a self-cleavage reaction in a β chain sequence specific manner. The cleavage fragment thereof or the activated ribozyme is then inspected to detect the β chain sequence. In general formulae (I) and (II), an enzyme site and a reaction site to be cleaved are present in the same chain (cis reaction). Also in general formula (III) in which the site to be cleaved is present in a chain apart from the enzyme, formation of a complex between β chain and γ chain induces the sequence specific cleavage of a ribonucleotide chain that can bind to the γ chain (trans reaction). The complex that is formed by β chain and γ chain at this time can enzymatically act on and cleave an excess amount of ribonucleotide chain. Thus, engineering of γ chain and β chain having structures represented by general formula (III) to cleave mRNA of a target gene that is causative of certain diseases can induce ribozyme activity for cleaving the target mRNA with the addition of β chain. Alteration of sequence combinations of β chain and γ chain further enables activity regulation on a plurality of activity-inducible ribozymes with the addition of a plurality of β chains, which are specific to respective ribozymes.

According to the present invention, a cleavage reaction that occurs in a sequence near 3′ terminus of ribozyme body by binding to an oligonucleotide is referred to as “self-cleavage”.

The ribozyme for detecting gene sequences according to the present invention effects self-cleavage when a subject oligonucleotide is present in a sample and the like. This reaction not only occurs in a test tube, but also has a potential to effect self-cleavage in a specific manner by binding to a polyribonucleotide having a subject nucleotide sequence produced in a cell. Thus, a polyribonucleotide with a subject sequence can be detected either in a plant, animal, or human body.

The present invention also provides DNA comprising a nucleotide sequence that is transcribed to the “ribozyme for detecting gene sequences” through RNA polymerase reaction, a recombinant vector comprising the DNA, and a host cell retaining the vector. Introducing the vector into a cell enables the cell to cleave a desired polyribonucleotide in a specific manner. The subject cell may be a plant, animal, or human body cell.

This specification includes part or all of the contents as disclosed in the specification and/or drawings of Japanese Patent Application No. 2000-399155, which is a priority document of the present application.

### BRIEF DESCRIPTION OF THE DRAWINGS

FIG. 1 shows the structure of a wild-type hairpin ribozyme.

FIG. 2 shows an example of ribozyme activation with an oligonucleotide.

FIG. 3a shows the structure of a wild-type hairpin ribozyme. Interaction between loop A and loop B results in cleavage at the site indicated with an arrow.

FIG. 3b shows a schematic diagram of a hairpin loop having a random sequence. The sequence expressed in lower-case letters is derived from template DNA, i.e., a sequence of a restriction site for cloning.

FIG. 4 shows a synthetic procedure for preparing DNA containing a nucleotide sequence transcribed into the ribozyme of the present invention.

FIG. 5 shows a procedure for the in vitro selection method used in the present invention.

FIG. 6 shows cleavage (%) and reaction time of negative and positive selection in each round of in vitro selection. Preselection 2 refers to a second preselection.

FIG. 7 shows predicted hairpin loop structures, upon binding to an oligonucleotide, of the respective clones having ribozyme activity according to the present invention.

FIG. 8 shows a three-dimensional change in stem-and-loop structure of the hairpin ribozyme according to the present invention, from an inactive conformation to an active conformation.

FIG. 9 shows a diagram for constructing template DNA encoding polyribonucleotide (1).

FIG. 10 shows an autoradiograph of polyribonucleotide (1) cleaved in the presence and absence of oligonucleotide (5).

FIG. 11 shows self-cleavage reaction effected in polyribonucleotide (1) during transcription from template DNA1 in the presence and absence of oligonucleotide (5).

### DETAILED DESCRIPTION OF THE INVENTION

The present invention will be described in more detail.

The present invention provides a hairpin ribozyme activated by changes in its stem-and-loop three-dimensional structure due to hybridization with an oligonucleotide. In the present specification, an “oligonucleotide” refers to an oligonucleotide that changes the stem-and-loop three-dimensional structure of the hairpin ribozyme according to the present invention, unless otherwise specified. The oligonucleotide may be a part of a longer nucleotide sequence although one with the chain length of 5 to 15 is preferably used.

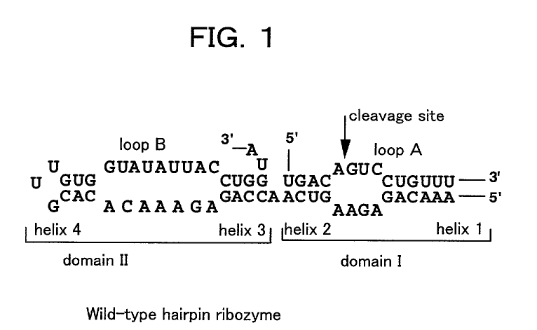

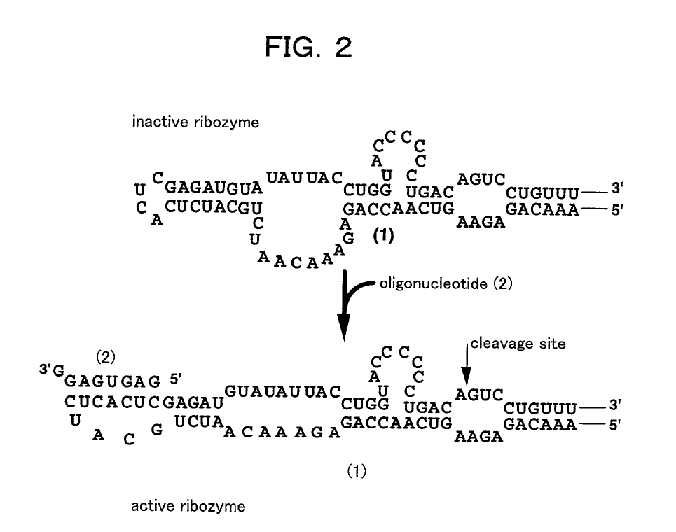

As described above, a ribozyme has a stem-and-loop structure as shown in FIG. 1, and it is known that the activity thereof is maintained by this three-dimensional structure. In the present invention, “changes in (its) stem-and-loop three-dimensional structure” refer to change, for example, described as the following: changes such that a nucleotide, that hybridizes intramolecularly and forms a stem and/or other nucleotides in the vicinity thereof (in the absence of an oligonucleotide), forms a loop because hybridization in the molecule is no longer possible due to hybridization with an oligonucleotide having a specific sequence. In such a case, a three-dimensional structure, which differs from the three-dimensional structure in the absence of the oligonucleotide, is adopted. This change in conformation is determined by which structure is more energetically stable: the three-dimensional structure of intramolecular hybridization in the absence of a oligonucleotide or the three-dimensional structure of hybridization with the oligonucleotide. Specifically, an oligonucleotide, which changes the stem-and-loop three-dimensional structure of a hairpin ribozyme, may be complementary to the sequence of the ribozyme to an extent that a complex, which is formed by hybridization with the ribozyme, has a more stable structure than forming intramolecular hybridization in the absence of the oligonucleotide. The hybridization formation between an oligonucleotide and a hairpin ribozyme suitable for activating the hairpin ribozyme according to the present invention changes depending on the domain sequence essential to cleavage activity of the ribozyme, and is in the range of 3 to 23 base pairs, preferably 5 to 15 base pairs, more preferably 7 to 12 base pairs.

In an embodiment of the present invention, an oligonucleotide is a target nucleotide sequence to be detected or a part thereof. In this embodiment, the oligonucleotide may be DNA or RNA, and one or more nucleotides may undergo modification such as methylation. In this embodiment, the presence or absence of the oligonucleotide, for example, in a sample or on a microarray such as a DNA chip can be detected by employing cleavage activity of the ribozyme as an index.

In this case, the ribozyme may be a cis-form ribozyme or a trans-form ribozyme. When a cis-form ribozyme is used, the ribozyme is activated by the presence of a target oligonucleotide and first effects self-cleavage. Suitable examples of such a cis-form ribozyme include those exhibiting a complex structure with an oligonucleotide according to general formula (I) or (II).

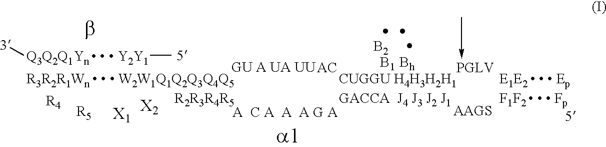

[wherein α1 represents a ribozyme sequence and β represents an oligonucleotide sequence:

in which U represents a uracil nucleotide, C represents a cytosine nucleotide, A represents an adenine nucleotide, and G represents a guanine nucleotide;

B1 to Bh, E1 to Ep, H1 to H4, Q1 to Q5, W1 to Wn, and X1 and X2, which may be the same or different, each represent any of a uracil nucleotide, an adenine nucleotide, a cytosine nucleotide, or a guanine nucleotide;

F1 to Fp, J1 to J4, R1 to R5, and Y1 to Yn each represent a nucleotide, which is complementary to E1 to Ep, H1 to H4, Q1 to Q5, and W1 to Wn, respectively;

S represents an adenine nucleotide or a cytosine nucleotide;

P represents any of a uracil nucleotide, an adenine nucleotide, a cytosine nucleotide, or a guanine nucleotide;

L represents any of a uracil nucleotide, an adenine nucleotide, or a cytosine nucleotide;

V represents an adenine nucleotide when S is a cytosine nucleotide and represents a uracil nucleotide or a cytosine nucleotide when S is an adenine nucleotide; and

h is an integer from 3 to 20, n is an integer from 1 to 10, and p is an integer from 1 to 10.]

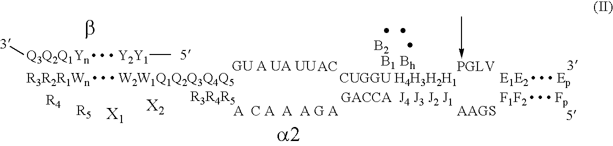

[wherein α2 represents a ribozyme sequence and β represents an oligonucleotide sequence with symbols used in this formula conveying the same meanings as used in general formula (I).]

In contrast, when a trans-form ribozyme is used, the ribozyme is activated by the presence of a target oligonucleotide, and cleaves other ribonucleotide sequences. Suitable examples of such trans-form ribozyme include ribozymes having a complex structure with an oligonucleotide in general formula (III).

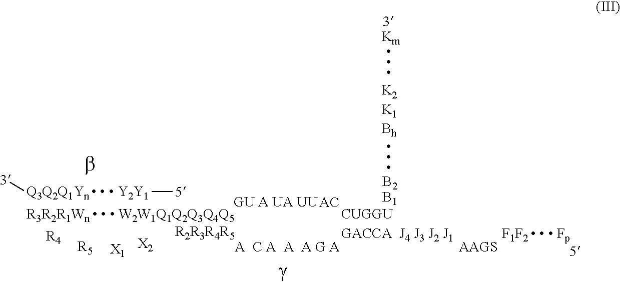

[wherein γ represents a ribozyme sequence and β represents an oligonucleotide sequence:

in which U represents a uracil nucleotide, C represents a cytosine nucleotide, A represents an adenine nucleotide, and G represents a guanine nucleotide;

B1 to Bh, F1 to Fp, J1 to J4, K1 to Km, Q1 to Q5, W1 to Wn, and X1 and X2, which may be the same or different, each represent any of a uracil nucleotide, an adenine nucleotide, a cytosine nucleotide, or a guanine nucleotide;

R1 to R5 and Y1 to Yn each represent a nucleotide, which is complementary to Q1 to Q5 and W1 to Wn, respectively;

S represents an adenine nucleotide or a cytosine nucleotide; and

h is an integer from 3 to 20, m is an integer from 1 to 10, n is an integer from 1 to 10, and p is an integer from 1 to 10.]

In this case, a ribonucleotide sequence to be cleaved depends on a sequence of a catalytic domain of the ribozyme and is a sequence satisfying conditions represented as a complex structure of an oligonucleotide and a trans-form ribozyme in general formula (IV). A person who has ordinary skill in the art can determine the sequence which can be cleaved in accordance with the ribozyme sequence and, alternatively, can properly engineer the ribozyme sequence for the predetermined ribonucleotide to be cleaved.

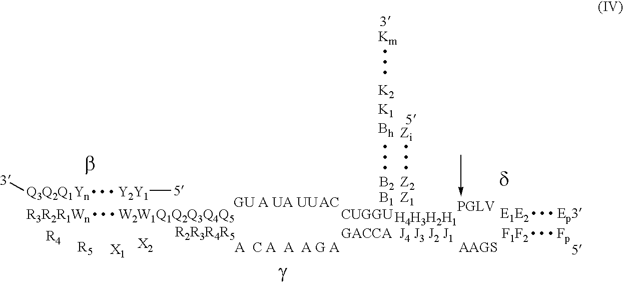

[wherein γ represents a ribozyme sequence, β represents an oligonucleotide sequence, and δ represents a ribonucleotide sequence to be cleaved:

in which Z1 to Zh (h≦i), E1 to Ep, and H1 to H4 each represent a nucleotide complementary to B1 to Bh, F1 to Fp, and J1 to J4, respectively;

P represents any of a uracil nucleotide, an adenine nucleotide, a cytosine nucleotide, or a guanine nucleotide;

L represents any of a uracil nucleotide, an adenine nucleotide, or a cytosine nucleotide;

V represents an adenine nucleotide when S is a cytosine nucleotide and represents a uracil nucleotide or a cytosine nucleotide when S is an adenine nucleotide; and

i is an integer the same as or larger than h; and

other symbols in this formula convey the same meanings as used in general formula (III)].

In general formulae (I), (II), and (III), h is generally 3 to 20, preferably 3 to 12, more preferably 3 to 8. Likewise, n is generally 1 to 10, preferably 3 to 8, more preferably 3 to 6; p is generally 1 to 10, preferably 3 to 10, more preferably 4 to 9; and m is generally 1 to 30, preferably 1 to 20, more preferably 2 to 5.

The cis-form hairpin ribozyme represented by general formulae (I) and (II) effects self-cleavage reaction at the site indicated with an arrow in the presence of an oligonucleotide. In contrast, the trans-form hairpin ribozyme represented by general formula (III) exhibits activity for cleaving another ribopolynucleotide in the presence of the oligonucleotide.

The present invention also provides DNA encoding a ribonucleotide which constitutes the above hairpin ribozyme, i.e., template DNA from which the ribonucleotide can be obtained via transcription.

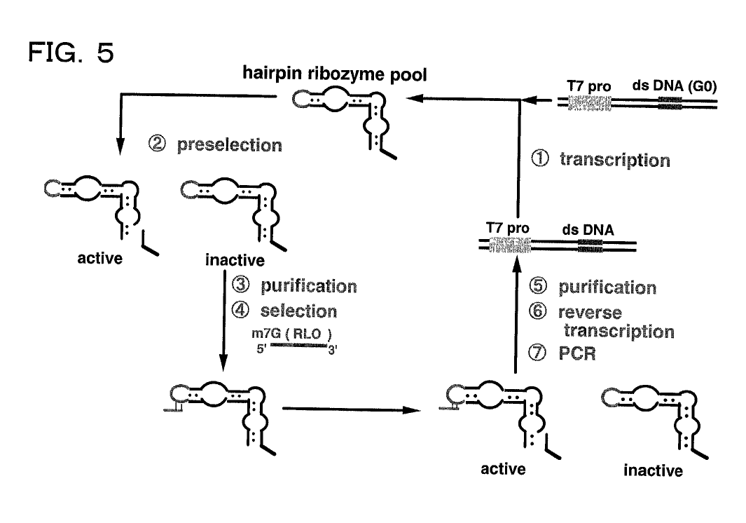

The hairpin ribozyme of the present invention may be constructed in the following manner, for example, by the in vitro selection method (FIG. 5).

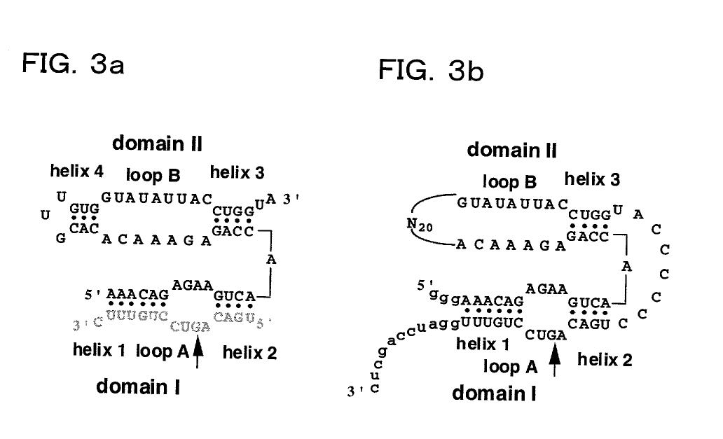

First, template DNAs are constructed to provide random nucleotide sequences for a hairpin loop region (N₂₀ region in FIG. 3b) of the hairpin ribozyme (4²⁰ types of template DNAs for ribozymes can be constructed because 19 to 20 nucleotides for the loop region are chosen at random), and a pool of ribozyme molecules is then obtained from the template DNAs via transcription. Thereafter, molecules showing cleavage activity by themselves are excluded (preselection or negative selection), and the remainder, which are inactive by themselves, are then subjected to a reaction in the presence of a target oligonucleotide to select molecules that effect cleavage reactions (positive selection; FIG. 5).

That is, the resulting RNA molecules are inactive by themselves, but are activated in the presence of the oligonucleotide. In fact, however, a single round of transcription, negative selection and positive selection cannot result in a real allosteric molecule. Hence, following positive selection, reverse transcription is preferably performed again to obtain template DNAs, each of which is then subjected to a second round of transcription, negative selection and positive selection. These procedures are preferably repeated to enrich active ribozymes.

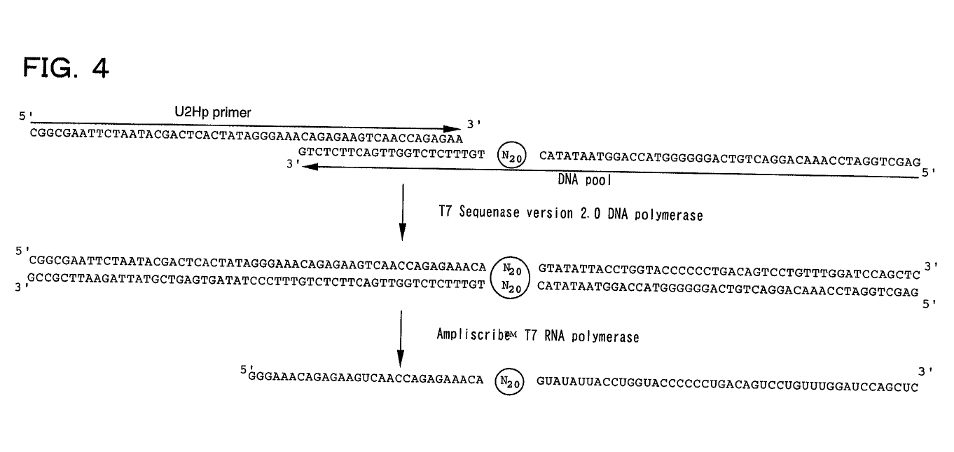

When a subject ribozyme sequence is previously known, or when the sequence is determined in accordance with the in vitro selection method, the ribozyme may be prepared by synthesis. For example, DNA comprising a nucleotide sequence, which is transcribed to the ribozyme according to the present invention, can be prepared by synthesizing a plurality of a sense and an antisense partial oligodeoxyribonucleotides so that sequences of the oligodeoxyribo nucleotides overlap at the both ends, then utilizing DNA polymerase reactions such as ligation by DNA ligase and polymerase chain reaction (PCR) [see Saiki, R. K. et al. (1988) Science 239, 487-491] to connect these partial oligodeoxyribonucleotides (FIG. 4).

A desired oligodeoxyribonucleotide sequence can be synthesized using a DNA/RNA automatic synthesizer (for example, manufactured by Applied Bio Systems Division, Perkin Elmer Japan Co., Ltd.), by using a nucleoside 3′-O-phosphoramidite in which a 5′-hydroxyl group is protected by a dimethoxytrityl group and an amino group in a base is protected by an acyl group (commercially available from Perkin Elmer, Amersham Pharmacia, or Glen Research) as a starting material [see Koster et al., (1984) Nucleic Acids Res. 12, 4539].

After completion of synthesis, β-cyanoethyl group as a protective group of a phosphate group is removed, a polynucleotide chain is excised from a carrier, and an acyl group in the base portion is removed with an alkali treatment, followed by an acid treatment to remove a protective group of a 5′-hydroxyl group. Subsequent purification, used in general purification of nucleic acid, for example, various types of chromatography such as reversed-phase and ion exchange chromatography (including high-performance liquid chromatography), can provide DNA chains. The nucleotide sequence of the obtained DNA can be confirmed by, for example, Maxam-Gilbert's chemical modification method [see Maxam, A. M. and Gilbert, W. (1980) Methods in Enzymology 65, 499-559] and the dideoxynucleotide chain termination method using M13 phage [see Messing, J. and Vieira, J. (1982) Gene 19, 269-276].

The sense chain and the antisense chain of the oligodeoxyribonucleotide thus obtained are annealed to prepare a double strand. The double-strand DNAs are then linked to a promoter that acts in a cell using a DNA ligase so that the DNAs are put under the control of the promoter. Hence, DNA, which is transcribed to the ribozyme of the present invention, can be constructed.

The ribozyme according to the present invention can be prepared, for example, by allowing T7 RNA polymerase to act on the above obtained DNA which is to be transcribed to the ribozyme of the present invention connected downstream of the promoter sequence of T7 RNA polymerase in the presence of ATP, CTP, GTP, and UTP [see Milligan, J. F. et al., (1987) Nucleic Acids Res. 15, 8783-8798]. In addition, other known RNA polymerase such as T3 RNA polymerase and SP6 RNA polymerase and other promoter sequences can be used in combination.

The thus obtained ribozyme according to the present invention can be used in the form of a salt. Such salts include inorganic or organic salts, for example, those with alkali metals such as sodium and potassium; those with alkaline earth metals such as calcium; those with ammonia; those with basic amino acids such as lysine and arginine; and those with alkylamines such as triethylamine.

The present invention further provides a recombinant vector containing DNA comprising a nucleotide sequence transcribed to the ribozyme according to the present invention, and a host cell to which the vector has been introduced. Introduction of the vector into a host cell provides the host cell of the present invention, and enables a mass production of expression vectors according to the present invention.

Examples of a host cell and vector include the following.

Examples of a procaryotic host cell include Escherichia coli and Bacillus subtilis. In order to express the subject genes in these host cells, the host cell may be subjected to transfection with a replicon derived from a variety compatible with the host cell, i.e., a plasmid vector containing a replication origin and a regulatory sequence. The vector preferably has a sequence in which a certain phenotype can be imparted to the transfected cell for selection

For example, E. coli K12 strain and the like are often used as Escherichia coli and, generally, pBR322 or pUC plasmid are often used as a vector. The present invention, however, is not limited to these, and various known strains and vectors may be used. Regarding promoters, a tryptophan (trp) promoter, a lactose (lac) promoter, a tryptophan-lactose (tac) promoter, a lipoprotein (lpp) promoter, a bacteriophage-derived λ PL promoter, and a polypeptide chain elongation factor Tu (tufB) promoter can be used in Escherichia coli. Any of these promoters may be used in the production of the ribozymes according to the present invention.

Further, a 207-25 strain is preferably used as Bacillus subtilis and pTUB28 [see Ohmura, K. et al., (1984) J. Biochem. 95, 87-93] and the like are preferably used as a vector, although the present invention is not limited to these. A regulatory sequence for α-amylase gene of Bacillus subtilis is often used as a promoter.

Cells of vertebrates, insects, yeasts and the like are applicable as eukaryotic host cells. For example, NIH-3T3 cell as mouse cell [see (1969) J. Viol. 4, 549-553], COS cell as monkey cell [see Gluzman, Y. (1981) Cell 23, 175-182], and dihydrofolate reductase deficient-strain of Chinese hamster ovarian cell (CHO) [see Urlaub, G. and Chasin, L. A., (1980) Proc. Natl. Acad. Sci. USA 77, 4216-4220] are often used as vertebrate cells, although the present invention is not limited to these. As expression vectors for vertebrate cells, vectors containing a promoter, which is generally located upstream of genes to be expressed, an RNA splice site, a polyadenylation site, a transcription termination site and the like may be used. They may have an origin of replication if necessary. Examples of expression vectors include pSV2dhfr having a SV40 early promoter [see Subramani, S. et al., (1981) Mol. Cell, Biol. 1, 854-864] and pcDL-SRα having a SRα promoter in which R-U5 of HTLV-I LTR is linked to the SV 40 early promoter [see Takebe, Y. et al., (1988) Mol. Cell Biol. 8, 466-472], although the present invention is not limited to these. Yeast may also be used as a eukaryotic microorganism. As an expression vector for a eukaryotic microorganism such as yeast, for example, those containing a promoter for alcohol dehydrogenase gene [see Bennetzen, J. and Hall, B. D.(1982) J. Biol. Chem. 257, 3018-3025] and a promoter for acid phosphatase gene [see Miyanohara, A. et al., (1983) Proc. Natl. Acad. Sci. USA 80,1-5] may be used.

Introduction of the thus obtained vector enables genetic transformation of host cells of other prokaryotic organisms or eukaryotic organisms. Further, genes can be expressed in each host cell by introducing an adequate promoter and a sequence associated with expression into these vectors.

For example, when Escherichia coli is employed as a host cell, an expression vector having a pBR322 origin of replication, capable of autonomous replication in Escherichia coli, and equipped with a transcriptional promoter and a translation initiation signal, can be used. The expression vector can be incorporated in Escherichia coli by, for example, calcium chloride method [see Mandel, M. and Higa, A. (1970) J. Mol. Biol. 53, 154], Hanahan's method [see Hanahan, D. and Meselson, M. (1980) Gene10, 63], and electroporation [see Neumann, E. et al., (1982) EMBO J. 1, 841-845]. This can provide a cell to which a desired vector is transfected.

When a COS cell is used, for example, an expression vector which has a SV 40 origin of replication, it is capable of autonomous replication in a COS cell, and is equipped with a transcriptional promoter, a transcription termination signal, and an RNA splice site, can be used. The expression vector can be incorporated in a COS cell by, for example, DEAE-dextran method [see Luthman, H. and Magnusson, G. (1983) Nucleic Acids Res. 11, 1295-1308], calcium phosphate-DNA co-precipitation method [see Graham, F. L. and van der Ed, A. J. (1973) Virology 52, 456-457], and electroporation [see Neumann, E. et al., (1982) EMBO J. 1, 841-845]. This can provide a cell to which a desired vector is transfected.

When a CHO cell is used as a host cell, co-transformation is carried out using an expression vector together with, for example, a vector that can express a neo gene functioning as a G418 resistant marker such as pRSV neo [see Sambrook, J. et al., (1989) “Molecular Cloning: A Laboratory Manual” Cold Spring Harbor Laboratory, NY] and pSV2-neo [see Southern, P. J. and Berg, P. (1982) J. Mol. Appl. Genet. 1, 327-341], and G418 resistant colonies are selected, thereby providing a transformed cell that stably produces the ribozyme according to the present invention.

The transformed cell thus obtained can be cultured in accordance with a conventional method, thereby producing the ribozyme according to the present invention in a cell. As a medium for the culture, various conventional media may be appropriately selected according to the adopted host cell, for example, in case of Escherichia coli, trypton-yeast medium (bacto-trypton 1.6%, yeast-extract 1.0%, NaCl 0.5% (pH 7.0)) and Pepton medium (DIFCO) may be used.

In case of a COS cell, a medium in which a blood serum component such as fetal bovine serum (FBS) may be optionally added to RPMI-1640 medium and Dulbecco's Modified Eagle Medium (DMEM) can be used.

Another embodiment of the present invention relates to a method for activating a hairpin ribozyme, comprising changing a stem-and-loop three-dimensional structure by hybridization of an oligonucleotide and an inactive ribozyme.

In this embodiment, an oligonucleotide may be a target nucleotide sequence, or it may be used as an activity regulator for a ribozyme. Specifically, a ribozyme, the activity of which has hitherto been difficult to regulate, can be activated by presence or absence of a specific oligonucleotide with particular sequence. In this case, an oligonucleotide may be an oligodeoxynucleotide or oligoribonucleotide. The oligonucleotide is preferably an oligoribonucleotide, and it is more preferred if one or more nucleotides are 2′-O-methylated.

The ribozyme according to the present invention changes so as to exhibit RNA cleaving activity in the presence of a particular oligonucleotide. This can be confirmed by, for example, the experiment described below.

Regarding a complex comprising two polyribonucleotide chains represented by α1 and β in general formula (I), the hairpin ribozyme α1 of the present invention and an oligonucleotide β to be detected are first heated in a buffer containing a divalent metal ion. Inspection of cleavage fragments of α1 enables detection of the presence of β chain. At this time, as a comparative experiment, a system in which an oligonucleotide β is not added is simultaneously tested. This experiment demonstrates that α1 is cleaved in a β sequence specific manner. Mg²⁺, Ca²⁺, Mn²⁺, Pb²⁺ and the like are preferably used as a divalent metal ion. Tris-HCl buffer, glycyl-glycine-sodium hydroxide buffer and the like may be used as a buffer, however, any buffer may be used so far as it can be used in a neutral or alkali condition without restriction.

Reaction temperature is preferably 0 to 100° C., more preferably 30 to 50° C.

After elapse of certain time, ethylenediaminetetraacetic acid (hereinafter referred to as “EDTA”) is added to the reaction solution to terminate the reaction.

A cleavage reaction using a complex consisting of α2 and β represented by general formula (II) is also performed in the same manner as in general formula (I).

The present invention also provides a method for detecting a target nucleotide sequence using a hairpin ribozyme according to the present invention. The target nucleotide sequence may be present in a sample or may be fixed on a microarray such as a DNA chip. In this embodiment, a cis-form ribozyme, which can self-cleave, is suitable although there is no particularly limitation. A suitable ribozyme includes, for example, those having a structure represented by general formula (I) or (II). In this case, self-cleavage of a ribozyme may be quantified by a method comprising labeling the ribozyme with a fluorochrome, a radioactive isotope and the like through transcription from template DNA and quantitatively analyzing a cleavage product of ribozyme produced with an externally-added oligonucleotide using a fluorescence microscope, an image analyzer, etc, or a method in which a cleavage product is quantitatively analyzed using reversed phase high performance liquid chromatography (HPLC).

The present invention further provides a kit for detecting a target nucleotide sequence in a sample comprising a ribozyme according to the present invention designed for a target RNA to be detected (corresponding to an oligonucleotide which can activate the ribozyme according to the present invention). In this case, in accordance with the nucleotide sequence of the target RNA to be detected, a nucleotide sequence of the ribozyme is previously designed to select the ribozyme for use depending on the target RNA. In the kit according to the present invention, in the case of a cis-form ribozyme, the ribozyme itself may be labeled with a fluorochrome, a radioactive isotope and the like and, in the case of a trans-form ribozyme, RNA, which is cleaved by a ribozyme, may be labeled with a fluorochrome, a radioactive isotope and the like. The presence of the subject RNA can be determined by mixing a ribozyme contained in the kit with a sample, activating the ribozyme with RNA in the sample, thereby effecting self-cleavage, and detecting the fluorescence intensity or a change in wavelength of the fluorochrome caused thereby.

The present invention further provides a kit for detecting a target nucleotide sequence in a sample, comprising a microarray such as a DNA chip and the hairpin ribozyme of the present invention. Accompanying progress in genome projects and developments in bioinformatics, a DNA chip is one means for analyzing nucleic acids, which has rapidly become utilized in recent years (Brown, P. O., Botstein, D. Nat Genet. 1999, 21, 33-37). In the kit of the present invention, various oligonucleotides, which are prepared from various samples, may be fixed on the DNA chip by known means in the art (Ramsay, G., Nature Biotech. 16, 40-44). A DNA chip (Hisahara, yeast, cDNA chip) onto which oligonucleotides are previously fixed may also be used. The ribozyme may be in a cis-form or trans-form, and cleavage fragments produced by self-cleavage or cleavage of other ribonucleotide sequences may be detected by the above-described detection method.

A still further embodiment of the present invention provides a method for cleaving a ribonucleotide sequence using the hairpin ribozyme of the present invention. According to this embodiment, a specific sequence in a sample can be cleaved. Further, the ribozyme can be administered to humans or animals as pharmaceuticals for prevention or treatment of diseases such as AIDS or cancer, which are caused by expression or overexpression of particular genes. In this case, the ribozyme according to the present invention may be administered concurrently with an oligonucleotide as an activity regulator, or may be administered separately from the oligonucleotide. The ribozyme, which cleaves a polyribonucleotide such as RNA in a sequence specific manner, is administered with an oligonucleotide that can regulate activity thereof, so that cleavage can also be regulated in a site-specific manner. As an oligonucleotide to regulate activity, an oligoribonucleotide is preferred and it is more preferred when one or more nucleotides thereof are 2′-O-methylated.

The present invention also provides a pharmaceutical composition for humans or animals, which contains the hairpin ribozyme according to the present invention.

The pharmaceutical composition according to the present invention may contain, for example, a carrier, a buffer, and a preservative that are pharmaceutically acceptable in addition to the ribozyme according to the present invention, and may contain other agents for subject diseases. Agents, which may be present together therewith, are not particularly limited unless they affect ribozyme activity. Dosage forms of the pharmaceutical composition of the present invention include those for oral administration, for example, a tablet, a capsule, a granule, a powder, and syrup and those for parenteral administration such as an injection, a drop, a suppository or the like.

The recombinant vector comprising DNA, which is transcribed to the ribozyme of the present invention, may be administered by encapsulation in a carrier such as a liposome. Examples of such liposomes include “Lipofectin” comprising N-[1-(2,3-dioleyloxy)propyl]-N,N,N-trimethylammonium chloride (DOTMA) and dioleoylphosphatidyl ethanolamine (DOPE) (manufactured by GIBCO BRL) and lipo-polyamine [see Behr, J. P. el al., (1989) Proc. Natl. Acad. Sci. USA 86, 6982-6986] although the liposome is not limited to these.

The dosage of the pharmaceutical composition according to the present invention varies depending on, for example, symptom, age, and weight of the subject. In general, for oral administration, a dosage is about 0.1 mg to 1,000 mg per day per adult, and this dose may be administered once or divided for several times. For parenteral administration, 0.1 mg to 1,000 mg per dose may be administered by subcutaneous injection, intramuscular injection, or intravenous injection.

The effect of the present invention may be attained by incorporating DNA of the present invention in a suitable expression vector, and administering the vector itself to a living organism, thereby expressing the ribozyme in a cell. Examples of such a vector include a retrovirus (for example, Moloney murine leukemic virus [see Eglitis, N. A. and Anderson, W. F. (1988) Biotechniques 6, 608-614]), an adenovirus [Yumi Kanegae et al., Experimental Medicine (1994) 12, 316-322], an adeno-associated virus [Yukihiko Hirai, Takashi Shimada, Experimental Medicine (1994) 12, 323-327], and a vaccinia virus, although it is not limited to these only.

### EXAMPLES

The present invention will be further described in the following examples and reference examples. These examples are provided for illustrative purposes only, and are not intended to limit the scope of the invention.

### Example 1

An oligonucleotide-responsive hairpin ribozyme was constructed (FIG. 3) by the in vitro selection method described below (FIG. 5).

First, template DNAs were constructed to provide random nucleotide sequences for a hairpin loop of the hairpin ribozyme (4²⁰ types of template DNAs for ribozymes can be constructed because 20 nucleotides for the loop region were chosen at random), and a pool of ribozyme molecules was then obtained from the template DNAs via transcription (FIG. 4). Thereafter, molecules showing cleavage activity by themselves were excluded (preselection or negative selection), and the remainder, which were inactive by themselves, were then subjected to a reaction in the presence of a target oligonucleotide to select molecules that effected cleavage reactions (positive selection; FIG. 5). That is, the resulting RNA molecules are inactive by themselves, but are activated in the presence of the oligonucleotide. In fact, however, a single round of transcription, negative selection and positive selection cannot result in a real allosteric molecule. Hence, following positive selection, reverse transcription was performed again to obtain template DNAs, each of which was then subjected to a second round of transcription, negative selection and positive selection. These procedures were repeated to enrich active ribozymes.

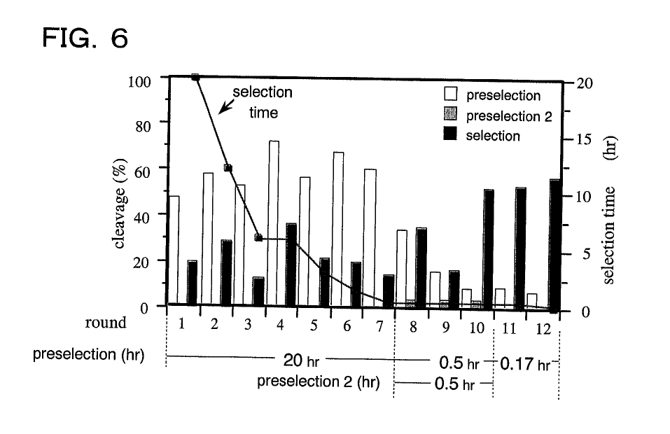

When a ribozyme having a sufficient allosteric effect was obtained by repeating several rounds, its template DNA was cloned and sequenced. FIG. 6 shows cleavage activity in each round of the negative and positive selection.

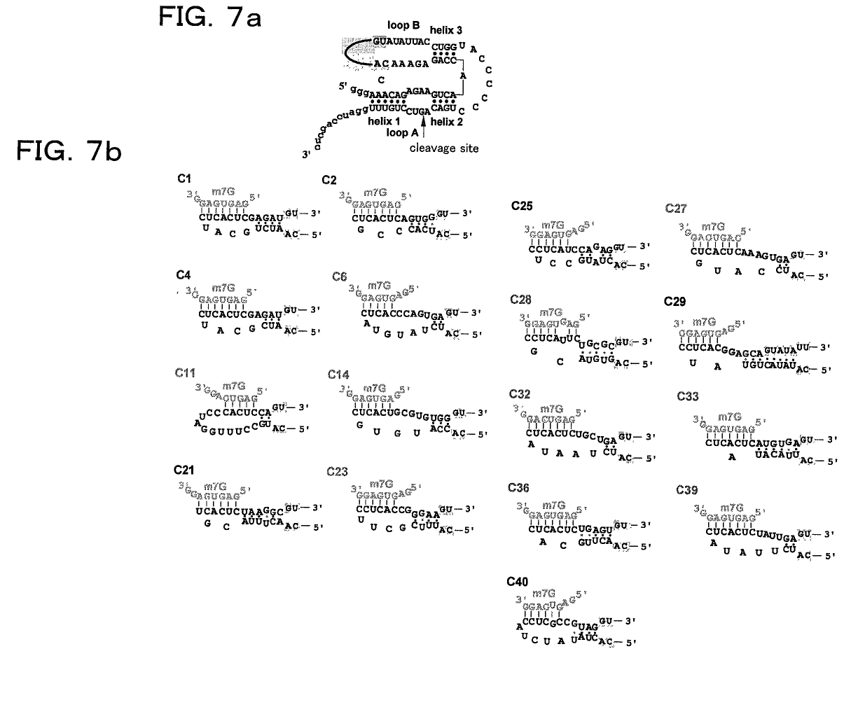

34 clones found to have an allosteric effect were sequenced, of which 18 clones shared the same sequence (C1), but the remainder were single clones (Table 1). When each clone was tested for its activity, it showed almost no activity in the absence of the oligonucleotide, while some of the clones were found to be activated approximately 200- to 300-fold by addition of the oligonucleotide (Table 1). The ribozyme sequence found in 18 clones (C1) also showed a high activation, and a ribozyme comprising a single nucleotide deletion in the C1 hairpin loop (C4) had a slightly higher activity than that of C1. FIG. 7 shows predicted hairpin loop structures of the respective clones upon binding to the oligonucleotide.

TABLE 1

| | | | m7G (+) | | |
|---|---|---|---|---|---|
| | Number | m7G (−) | | End point | k_{obs(+)}/ |
| Clone | of clones | k_{obs(-)} (min⁻¹) | k_{obs(+)} (min⁻¹) | (%) | k_{obs(-)} |
| C1 | 18 | 0.0033 | 0.69 | 63 | 207 |
| C2 | 1 | 0.0019 | 0.66 | 67 | 345 |
| C4 | 1 | 0.0023 | 0.74 | 67 | 327 |
| C6 | 1 | 0.0039 | 0.20 | 61 | 51 |
| C11 | 1 | 0.035 | 0.28 | 64 | 7.9 |
| C14 | 1 | 0.0031 | 0.25 | 68 | 80 |
| C21 | 1 | 0.0022 | 0.12 | 65 | 53 |
| C23 | 1 | 0.0026 | 0.34 | 69 | 133 |
| C25 | 1 | 0.0018 | 0.15 | 63 | 87 |
| C27 | 1 | 0.0021 | 0.25 | 62 | 120 |
| C28 | 1 | 0.0022 | 0.56 | 69 | 257 |
| C29 | 1 | 0.0030 | 0.14 | 58 | 46 |
| C32 | 1 | 0.0026 | 0.55 | 69 | 208 |
| C33 | 1 | 0.0038 | 0.55 | 73 | 143 |
| C36 | 1 | 0.0024 | 0.54 | 69 | 223 |
| C39 | 1 | 0.0028 | 0.53 | 71 | 190 |
| C40 | 1 | 0.0018 | 0.054 | 46 | 30 |
| Total | 34 | | | | |

The oligonucleotide used in this in vitro selection is 2′-O-methyl-oligonucleotide (m7G 2′-OMe(GAGUGAG)rG; only the 3′-terminal G is a ribonucleotide). However, C1 also showed a sufficiently high activity in the presence of a non-methylated oligoribonucleotide (r7G).

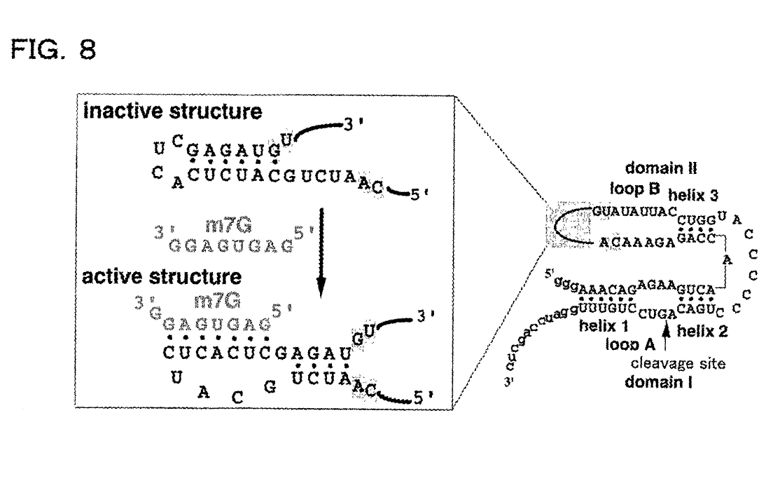

These results indicated a system in which internal loop B (FIG. 3) required for activity of a hairpin ribozyme is unable to form an active conformation in the absence of an oligonucleotide, but the ribozyme is activated as the internal loop B is converted to an active conformation when the oligonucleotide binds to the hairpin loop. That is, a sequence inducing a conformational change as shown in FIG. 8 would be interesting not only in respect of the activation of a hairpin ribozyme, but also as a module for transmitting the sequence to an enzymatic site.

### Example 2

### Synthesis of Oligodeoxyribonucleotide

Template DNA1 (SEQ ID NO: 1) and template DNA2 (SEQ ID NO: 3) were synthesized for use in transcription to obtain polyribonucleotide (1) (SEQ ID NO: 2) and polyribonucleotide (2) (SEQ ID NO: 4) having the following structures, and whose cleavage activity is induced by an oligoribonucleotide.

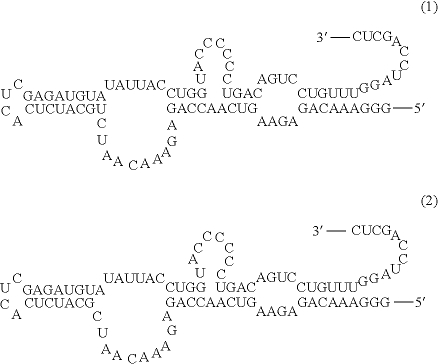

First, the following oligodeoxyribonucleotides were synthesized:

5′-CGGCGAATTCTAATACGACTCACTATAGGGAAACAGAGAAGTCAA CCAGAGAA (U1; SEQ ID NO: 5);

GAGCTGGATCCAAACAGGACTGTCAGGGGGGTACCAGGTAATATACA TCTCGAGTGAGATGCAGATTGTTTCTCTGGTTGACTTCTCTG (L1; SEQ ID NO: 6); and

GAGCTGGATCCAAACAGGACTGTCAGGGGGGTACCAGGTAATATACA TCTCGAGTGAGATGC GATTGTTTCTCTGGTTGACTTCTCTG (L2; SEQ ID NO: 7).

Template DNA1 was prepared from U1 and L1, while template DNA2 was prepared from U1 and L2.

Each of the above oligodeoxyribonucleotides was synthesized in an automatic DNA synthesizer (model 394A; Perkin Elmer Japan, Applied Biosystems Division) using deoxynucleoside 3′-phosphoramidite (purchased from Perkin Elmer Japan, Applied Biosystems Division) as a starting material. The synthesis was performed at a scale of 1 μmol.

After completion of synthesis, U1 was treated and purified as follows. The oligodeoxyribonucleotide was excised from CPG (Controlled Pore Glass) with concentrated aqueous ammonia, and heated at 60° C. for 5 hours. After distillation of the solvent, the resulting residue was dissolved in deionized water and applied to column chromatography on C18 silica gel (Waters Corporation) under the following conditions: a column size of 1.5×12 cm; eluted with a linear gradient of 5-40% acetonitrile in 0.1 M aqueous triethylammoniumacetate (hereinafter, referred to as “TEAA”). Fractions developing the color of dimethoxytrityl, which were eluted with about 30% acetonitrile, were collected and stirred with 2 ml of 80% aqueous acetic acid for 1 hour. Acetic acid was distilled off under reduced pressure, and the aqueous phase was washed with ethyl acetate. After distillation of the solvent, the resulting residue was dissolved in 1 ml of sterilized water. Oligoribonucleotides present in this fraction were collected by reversed-phase HPLC, followed by ion-exchange HPLC, to purify the product. Conditions used for reversed-phase HPLC are presented below:

Column: μ-Bondasphere (C-18) column, Φ3.9×150 mm (Waters Corporation);

Solvent: Solution A—5% acetonitrile/0.1 M TEAA (pH 7.0); and

Solution B—25% acetonitrile/0.1 M TEAA (pH 7.0).

Table 2 shows linear gradient conditions used for reversed-phase HPLC of U1, along with the retention time thereof.

TABLE 2

| | | Total time required for elution with | |
|---|---|---|---|
| | B % | linear gradient | Retention time |
| U1 | 20 → 40% | 20 minutes | 16.2 minutes |

L1 and L2 were synthesized in the automatic DNA synthesizer in the same manner as U1, and then electrophoresed on a 8% denatured polyacrylamide gel, followed by ultraviolet irradiation at 254 nm and excision of a gel slice containing L1 and L2 of interest. The gel slice was immersed in 800 μl of sterilized water to extract a nucleic acid from the gel slice. The extracted nucleic acid was then purified by desalting with NAP10 (Amersham Pharmacia Biotech) to obtain L1 and L2.

### Example 3

### Preparation of Template DNAs

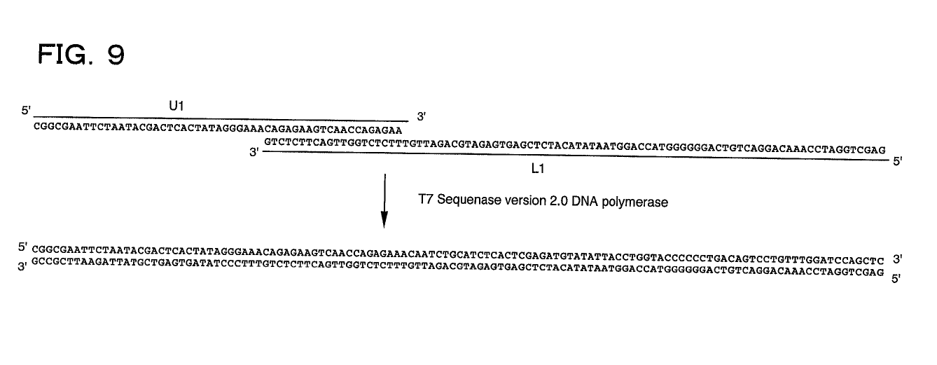

Template DNA1 was constructed as follows. FIG. 9 shows a construction scheme.

L1 DNA (200 pmol) and U1 primer (240 pmol) were added with 20 μl of T7 sequenase reaction buffer (×5, 200 mM Tris-HCl (pH 7.5), 100 mM MgCl_{2,} 250 mM NaCl), adjusted to a total volume of 93 μl with sterilized water, denatured at 90° C. for 5 minutes, and then slowly cooled to room temperature. The mixture was then added with 0.1 M dithiothreitol (5 μl; hereinafter, referred to as “DTT”), 25 mM dATP, 25 mM dCTP, 25 mM dGTP and 25 mM dTTP (1.5 μl each), and T7 sequenase DNA polymerase (0.5 μl, 6.5U; Amersham Pharmacia Biotech), and extension reaction was performed at 37° C. for 2 hours. This reaction solution was subjected to phenol treatment and then phenol-chloroform treatment, followed by ethanol precipitation. The collected template DNA was dissolved in 20 μl, of TE buffer. The resulting template DNA was separated and purified on a 10% non-denatured polyacrylamide gel (29:1). The collected template DNA was dissolved in 30 μl of 10 mM Tris-HCl buffer (pH 8.0).

Template DNA2 was also prepared in the same manner as template DNA1.

### Example 4

### Transcription of Polyribonucleotide (1) from Template DNA1

Template DNA1 prepared in Example 3 was used to obtain polyribonucleotide (1) of interest via transcription described below. Transcription was performed using an AmpliScribe™ T7 transcription kit (AR BROWN Co., Ltd.). Template DNA1 or DNA2 (20 ng), T7 transcription buffer (1 μl; ×10; included in the kit), 25 mM ATP, 25 mM CTP, 25 mM GTP and 25 mM UTP (3 μl each), [α−³²P] UTP (0.5 μl), 100 mM DTT (1 μl) and AmpliScribe™ T7 enzyme solution (1 μl; included in the kit) were mixed together and adjusted to a total volume of 10 μl with sterilized water, and then reacted at 37° C. for 2 hours. The reaction mixture was treated with 0.5 μl of RNase-free DNase I (1 molecular biology unit) at 37° C. for 15 minutes and then roughly purified with NAP5 (Amersham Pharmacia Biotech). The resulting product was subjected to phenol-chloroform treatment and then chloroform treatment, followed by ethanol precipitation. The collected RNA was dissolved in 20 μl of sterilized water.

Template DNA2 was also used for transcription in the same manner as template DNA1 to obtain polyribonucleotide (2).

Polyribonucleotide (1) corresponds to α1 in the claimed general formula (I), while polyribonucleotide (2) corresponds to α2 in the claimed general formula (II).

### Reference Example 1

### Synthesis of Oligoribonucleotides Used for Induction of Ribozyme Activity

Oligoribonucleotide (3) (GAGUGAGG) which is capable of binding to hairpin loops of polyribonucleotides (1) and (2), as well as oligoribonucleotide (4) (GAGUGUCG) designed such that 2 nucleotides in the sequence of oligoribonucleotide (3) are replaced by nucleotides not complementary to polyribonucleotide (1) were synthesized, collected and purified as follows. Oligoribonucleotide (4) was synthesized as a less base-pairing oligoribonucleotide in order to confirm the specific activation of polyribonucleotides (1) and (2) with oligoribonucleotide (3).

Each of oligoribonucleotides (3) and (4) was synthesized in an automatic DNA synthesizer (Applied Biosystem Model 394A) using ribonucleoside 3′-phosphoramidite (GLEN Research). Both RNA fragments were synthesized at a scale of 1 μmol. After completion of synthesis, CPG (Controlled Pore Glass) containing the synthesized oligonucleotide bound thereto was treated with a mixture of concentrated aqueous ammonia and ethanol (3:1 v/v) at room temperature for 2 hours, and heated at 55° C. for 16 hours. The solvent was distilled off and the resulting residue was stirred with 1 ml of 1 M TBAF (tetrabutylammonium fluoride)/THF (tetrahydrofuran) solution at 37° C. for 16 hours. After adding 5 ml of 0.1 M triethylammonium acetate (pH 7.0), the mixture was applied to column chromatography on C18 silica gel (Waters Corporation) under the following conditions: a column size of 1.5×12 cm; eluted with a linear gradient of 5-40% acetonitrile in 50 mM aqueous triethylammonium bicarbonate. Fractions developing the color of dimethoxytrityl, which were eluted with about 30% acetonitrile, were collected and stirred with 5 ml of 0.01 N HCl for 1 hour. After neutralizing with 0.1 N aqueous ammonia, the aqueous phase was washed with ethyl acetate. The solvent was distilled off and the resulting residue was dissolved in 1 ml of sterilized water. Oligoribonucleotides present in this fraction were collected by reversed-phase HPLC, followed by ion-exchange HPLC, to purify the product. The oligonucleotides thus obtained were provided for subsequent experiments described below, in order to be used in a ribozyme cleavage assay. Oligonucleotide (4) was purified by ion-exchange HPLC alone, without combination with reversed-phase HPLC, resulting in a high purity.

Conditions used for reversed-phase HPLC and ion-exchange HPLC are presented below:

Reversed-phase HPLC

Column: μ-Bondasphere (C-18) column, Φ3.9×300 mm (Waters Corporation);

Solvent: Solution A—5% acetonitrile/0.1 M TEAA (pH 7.0); and

Solution B—25% acetonitrile/0.1 M TEAA (pH 7.0)

Ion-exchange HPLC

Column: TSKgel DEAE 2SW column, Φ4.6×250 mm (TOSOH Corporation);

Solvent: Solution A—20% acetonitrile; and

Solution B—2M HCOONH₄ containing 20% acetonitrile

Table 3 shows linear gradient conditions used for reversed-phase HPLC and ion-exchange HPLC of oligoribonucleotides (3) and (4), along with the retention time thereof.

TABLE 3

| | | |
|---|---|---|
| | Total time re- | |
| | quired for elu- | |
| | tion with linear | |
| B % | gradient | Retention time |

Reversed-phase HPLC

| | | | |
|---|---|---|---|
| Oligonucleotide (3) | 0 → 20% | 20 minutes | 19 minutes |

Ion-exchange HPLC

| | | | |
|---|---|---|---|
| Oligonucleotide (3) | 15 → 35% | 20 minutes | 17 minutes |
| Oligonucleotide (4) | 15 → 35% | 20 minutes | 16 minutes |

### Reference Example 2

### Synthesis and Purification of 2′-O-Methyloligonucleotide (5) (GAGUGAGG)

2′-O-methyloligoribonucleotide (5), which has the same sequence as oligonucleotide (3), was synthesized and purified in the same manner as Reference Example 1 in an automatic DNA synthesizer (Applied Biosystem Model 394A) using 2′-O-methylribonucleoside 3′-phosphoramidite (GLEN Research) instead of ribonucleoside 3′-phosphoramidite (GLEN Research) used for the synthesis in Reference Example 1 above. However, 2′-O-methyloligoribonucleotide (5) was synthesized to have a normal ribonucleoside at its 3′-terminus by using a ribonucleoside resin. That is, the synthesized 2′-O-methyloligoribonucleotide (5) has the structure: 5′[2′-OMe(GAGUGAG)rG]3′. Reversed-phase HPLC and ion-exchange HPLC were performed using the same column and solvent as used in Reference Example 1. Table 4 shows linear gradient conditions used for reversed-phase HPLC and ion-exchange HPLC of oligoribonucleotide (5), along with the retention time thereof.

TABLE 4

| | | |
|---|---|---|
| | Total time re- | |
| | quired for elu- | |
| | tion with linear | |
| B% | gradient | Retention time |

Reversed-phase HPLC

| | | | |
|---|---|---|---|
| Oligonucleotide (5) | 15 → 35% | 20 minutes | 12.7 minutes |

Ion-exchange HPLC

| | | | |
|---|---|---|---|
| Oligonucleotide (5) | 15 → 35% | 20 minutes | 17.5 minutes |

### Example 5

### Cleavage Assay on Polyribonucleotide (1) in the Presence and Absence of Oligonucleotide

Polyribonucleotide (1) obtained in Example 4 (16 pmol) was dissolved in 8 μl of cleavage buffer (×1; 40 mM Tris-HCl (pH 7.5), 12 mM magnesium chloride, 2 mM spermidine trihydrochloride). In addition, oligonucleotide (3), (4) or (5) (200 pmol) was dissolved in 10 μl of cleavage buffer. After denaturing at 90° C. for 2 minutes, polyribonucleotide (1) was cooled on ice and then mixed with 8 μl of previously heat-denatured oligonucleotide (3), (4) or (5) to start the reaction at 37° C. An aliquot was sampled at given time intervals and added into a solution containing 50 mM EDTA and 10 M urea (5 μl) to stop the reaction. The resulting reaction mixture was electrophoresed on a 20% polyacrylamide gel containing 8 M urea to separate reacted and unreacted products. The electrophoresed gel was analyzed using a bioimage analyzer (BAS 2000; Fuji Photo Film Co., Ltd.). More specifically, the reacted and unreacted products were radioactively quantified to determine the percentage of cleavage, thereby calculating an apparent reaction rate constant k_{obs}. Also, the cleavage assay on polyribonucleotide (1) in the absence of an oligonucleotide was performed by adding 8 μl of the above cleavage buffer (×1) alone, without addition of the above oligoribonucleotide (3), (4) or (5). An aliquot was sampled at given time intervals to determine the percentage of cleavage, from which a reaction rate constant was calculated.

An RNA hybrid formed between polyribonucleotide (1) and oligonucleotide (3) is encompassed in the activated ribozyme for detecting gene sequences according to the present invention. Polyribonucleotide (1) effected no cleavage reaction by itself, but cleaved itself at the site indicated with an arrow upon binding to oligonucleotide (3).

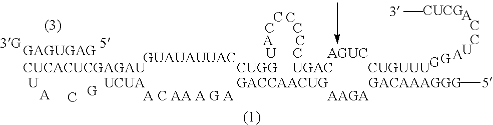

An RNA hybrid formed between polyribonucleotide (1) and oligoribonucleotide (4) was unstable due to relatively weak hydrogen binding between these two RNA molecules, which effected almost no cleavage reaction in polyribonucleotide (1). This is because oligoribonucleotide (4) is not completely complementary to polyribonucleotide (1), i.e., only five 5′-terminal nucleotides of oligoribonucleotide (4) are complementary to polyribonucleotide (1).

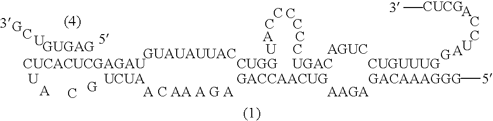

An RNA hybrid formed between polyribonucleotide (1) and oligoribonucleotide (5) satisfies the structure defined in the present invention, wherein seven 5′-terminal nucleotides of RNA corresponding to β in the claimed general formula (I) are replaced by 2′-O-methyloligoribonucleotide. A hybrid of 2′-O-methyl-oligoribonucleotide and RNA is known to form a more stable duplex as compared to an RNA/RNA hybrid. Accordingly, 2′-O-methyloligoribonucleotide was tested for its ability to induce the cleavage activity of polyribonucleotide (1).

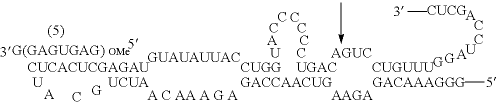

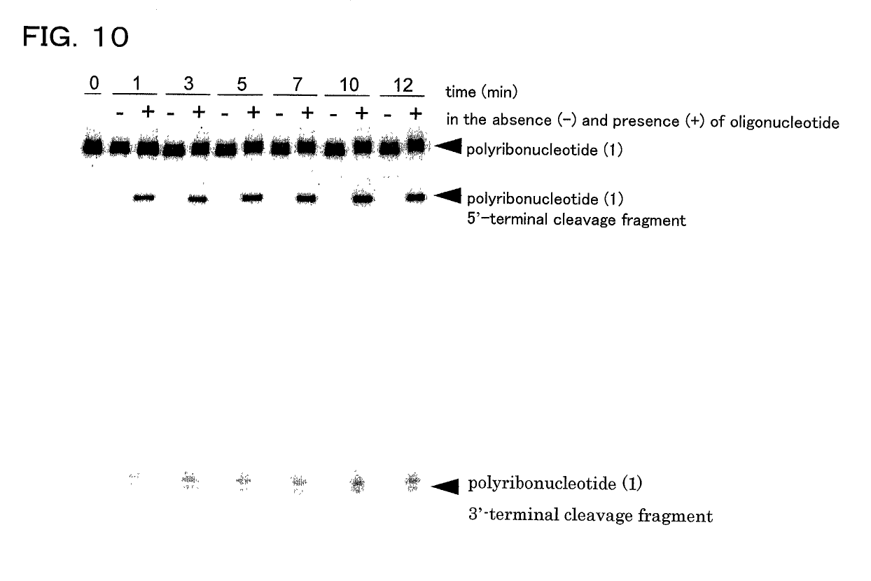

Polyribonucleotide (1) effected almost no self-cleavage reaction in the absence of oligonucleotide (3) and in the presence of oligonucleotide (4) which cannot stably bind to polyribonucleotide (1), whereas it specifically effected self-cleavage reaction in the presence of oligonucleotide (3) which is complementary to polyribonucleotide (1). This suggests that polyribonucleotide (1) specifically cleaved itself in the presence of an oligonucleotide capable of binding to polyribonucleotide (1), thereby allowing sequence detection. In addition, since polyribonucleotide (1) also showed a high cleavage activity by addition of oligonucleotide (5), it was found that not only RNA strands, but also 2′-O-methyloligoribonucleotide molecules capable of stably binding to a ribozyme can induce the activity of polyribonucleotide (1). By way of example, FIG. 10 shows denatured polyacrylamide gel electrophoresis of polyribonucleotide (1) cleaved for 0, 1, 3, 5, 7, 10 and 12 minutes in the presence (+ lanes) or absence (− lanes) of oligonucleotide (5). Polyribonucleotide (1) results in no cleavage fragment by itself, but effects a specific cleavage reaction when oligonucleotide (5) is added thereto.

Table 5 shows cleavage rate constants (k) of polyribonucleotide (1) in the presence of the respective oligonucleotides. 2′-O-methyloligoribonucleotide (5) induced the cleavage activity of polyribonucleotide (1) most efficiently. This would be because 2′-O-methyloligoribonucleotide can bind to RNA more stably than a normal oligonucleotide.

TABLE 5

| | k |
|---|---|
| Polyribonucleotide (1) | 0.0033 min⁻¹ |
| Polyribonucleotide (1) + oligonucleotide (3) | 0.61 min⁻¹ |
| Polyribonucleotide (1) + oligonucleotide (4) | 0.0022 min⁻¹ |
| Polyribonucleotide (1) + oligonucleotide (5) | 0.69 min⁻¹ |

(N.D.: not detected)

### Example 6

### Cleavage Assay on Polyribonucleotide (2) in the Presence and Absence of Oligonucleotide

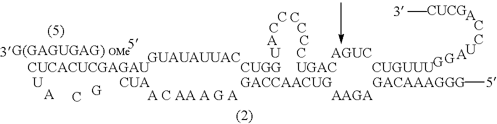

In the same manner as used for cleavage assay in Example 5, polyribonucleotide (2) obtained in Example 4 was tested for its cleavage activity by addition of an oligonucleotide. As an oligonucleotide, 2′-O-methyloligoribonucleotide (5) was used, which was found to have the highest induction activity in Example 5. Polyribonucleotide (2) (16 pmol) was dissolved in 8 μl of cleavage buffer (×1; 40 mM Tris-HCl (pH 7.5), 12 mM magnesium chloride, 2 mM spermidine trihydrochloride). In addition, oligonucleotide (5) (200 pmol) was dissolved in 10 μl of cleavage buffer. After denaturing at 90° C. for 2 minutes, polyribonucleotide (2) was cooled on ice and then mixed with 8 μl of previously heat-denatured oligonucleotide (5) to start the reaction at 37° C. An aliquot was sampled at given time intervals and added into a solution containing 50 mM EDTA and 10 M urea (5 μl) to stop the reaction. The resulting reaction mixture was electrophoresed on a 20% polyacrylamide gel containing 8 M urea to separate reacted and unreacted products. The electrophoresed gel was analyzed using a bioimage analyzer (BAS 2000; Fuji Photo Film Co., Ltd.). More specifically, the reacted and unreacted products were radioactively quantified to determine the percentage of cleavage, thereby calculating an apparent reaction rate constant k_{obs} (Table 6). Also, the cleavage assay on polyribonucleotide (2) in the absence of an oligonucleotide was performed by adding 8 μl of the above cleavage buffer (×1) alone, without addition of the above oligoribonucleotide (5). An aliquot was sampled at given time intervals to determine the percentage of cleavage, from which a reaction rate constant was calculated.

Polyribonucleotide (2) effected almost no cleavage reaction by itself, but cleaved itself at the site indicated with an arrow in the presence of oligonucleotide (5).

TABLE 6

| | k |
|---|---|
| Polyribonucleotide (2) | 0.0023 min⁻¹ |
| Polyribonucleotide (2) + oligonucleotide (5) | 0.74 min⁻¹ |

N.D.: not detected)

### Example 7

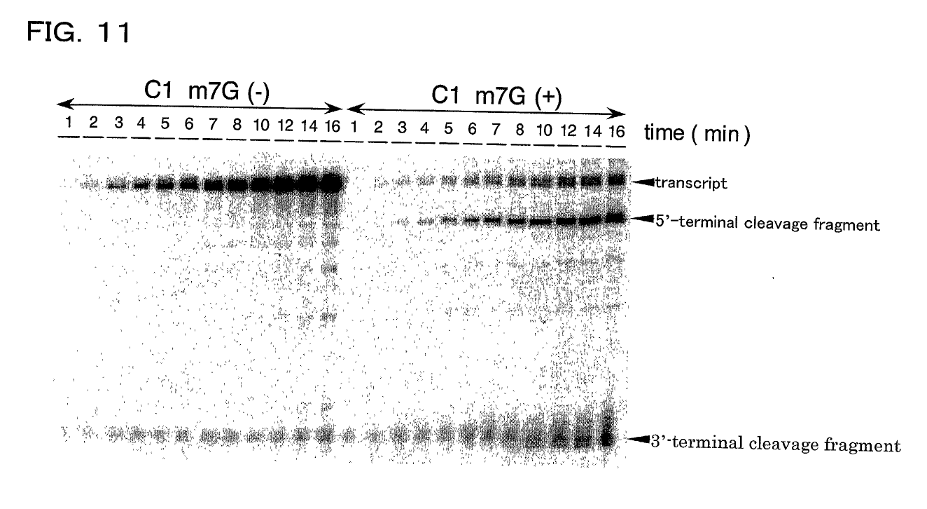

In Examples 5 and 6, a ribozyme transcribed from template DNA was first isolated and then assayed for its cleavage activity in the presence of an oligonucleotide. In such a case, however, the amount of the resulting cleavage products is limited by the initial amount of RNA provided for the assay. In addition, when intended to be used as a detection reagent, RNA may also have difficulties associated with storage etc. because of its instability. In order to increase detection sensitivity and further investigate whether a ribozyme effects cleavage reaction without isolation of the transcribed RNA, the cleavage assay was performed during transcription from template DNA. Ribozyme molecules transcribed one after another from template DNA may effect cleavage reactions upon binding to an oligonucleotide coexistent therewith and thus can be expected to result in more cleavage products and a higher sensitivity. Template DNA1 or template DNA2 (50 nmol) and oligoribonucleotide (5) (600 pmol) were dissolved in 4 μl of cleavage buffer (×5; 200 mM Tris-HCl (pH 7.5), 60 mM magnesium chloride, 10 mM spermidine trihydrochloride), 4 μl of 2.5 mM NTPs (ATP, GTP, CTP, UTP), 1 μl of [α−³²P]UTP, 1 μl of 100 mM DTT and an appropriate amount of sterilized water. 2 μl of Ampliscribe T7 RNA polymerase was added to the solution in a total volume of 20 μl to start the reaction at 37° C. An aliquot (1.5 μl) was sampled at appropriate time points and added into a solution containing 50 mM EDTA and 10 M urea (5 μl) to stop the reaction. The resulting reaction mixture was electrophoresed on a 10% polyacrylamide gel containing 8 M urea to separate reacted and unreacted products. The electrophoresed gel was analyzed using a bioimage analyzer (BAS 2000; Fuji Photo Film Co., Ltd.). FIG. 11 shows gel electrophoresis of polyribonucleotide (1) specifically cleaved during transcription in the presence of oligoribonucleotide (5). More specifically, the reacted and unreacted products were radioactively quantified to determine the percentage of cleavage, thereby calculating an apparent reaction rate constant k_{obs}.

Polyribonucleotide (1) or (2) showed almost no cleavage activity in the absence of oligoribonucleotide (5), whereas it was observed to effect a significant cleavage reaction in the presence of oligoribonucleotide (5).

TABLE 7
Reaction rate constants of self-cleavage during transcription

| | k |
|---|---|
| Polyribonucleotide (1) | N.D. |
| Polyribonucleotide (1) + oligonucleotide (5) | 0.57 min⁻¹ |
| Polyribonucleotide (2) + oligonucleotide (5) | 0.78 min⁻¹ |

(N.D.: not detected)

Industrial Applicability

The activity-inducible hairpin ribozyme constructed herein is a novel ribozyme showing self-cleavage activity upon specifically recognizing a particular RNA sequence. The ribozyme has a recognition site different from its catalytic site, which is located at a distance from the catalytic site. In the absence of target RNA externally added, the ribozyme takes an inactive conformation, thereby showing no activity. However, in the presence of the target RNA, the ribozyme undergoes a conformational change upon binding to the RNA to take an active conformation, thereby inducing cleavage activity. This novel ribozyme may be regarded as an RNA enzyme with sensor functions because it shows catalytic activity upon sequence-specific recognition of a particular target RNA. Thus, a ribozyme constructed to specifically distinguish mRNA having a nucleotide sequence of interest allows conversion of the gene sequence into signals of RNA cleavage by the ribozyme, and may therefore be applied to detection of genetic polymorphism. The present invention provides a novel method for detecting gene expression without the need for immobilization and PCR which are necessary for conventional detection methods using immobilized genes. Accordingly, the detection method of the present invention is expected to be a more convenient detection method.

All publications, patents and patent applications cited herein are incorporated herein by reference in their entirety.

What is claimed is:

1. A hairpin ribozyme activated by changes in its stem-and-loop three-dimensional structure due to hybridization with an oligonucleotide.

2. The hairpin ribozyme according to claim 1, wherein the hybridization with an oligonucleotide is constituted by 3 to 23 base pairs.

3. The hairpin ribozyme according to claim 1, wherein the oligonucleotide is a part of a target nucleotide sequence.

4. The hairpin ribozyme according to claim 1, which is a cis-form ribozyme which first self-cleaves upon activation.

5. The hairpin ribozyme according to claim 4, which exhibits a complex structure with an oligonucleotide as shown in general formula (I) or (II) 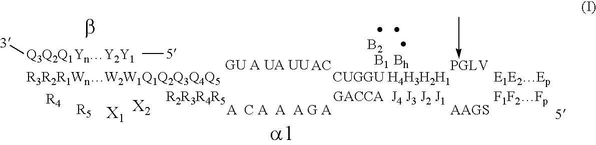
[wherein α1 represents a ribozyme sequence and β represents an oligonucleotide sequence:
in which U represents a uracil nucleotide, C represents a cytosine nucleotide, A represents an adenine nucleotide, and G represents a guanine nucleotide;
B1 to Bh, E1 to Ep, H1 to H4, Q1 to Q5, W1 to Wn, and X1 and X2, which may be the same or different, each represent any of a uracil nucleotide, an adenine nucleotide, a cytosine nucleotide, or a guanine nucleotide;
F1 to Fp, J1 to J4, R1 to R5, and Y1 to Yn each represent a nucleotide, which is complementary to E1 to Ep, H1 to H4, Q1 to Q5, and W1 to Wn, respectively;
S represents an adenine nucleotide or a cytosine nucleotide;
P represents any of a uracil nucleotide, an adenine nucleotide, a cytosine nucleotide, or a guanine nucleotide;
L represents any of a uracil nucleotide, an adenine nucleotide, or a cytosine nucleotide;
V represents an adenine nucleotide when S is a cytosine nucleotide and represents a uracil nucleotide or a cytosine nucleotide when S is an adenine nucleotide; and
h is an integer from 3 to 20, n is an integer from 1 to 10, and p is an integer from 1 to 10.] 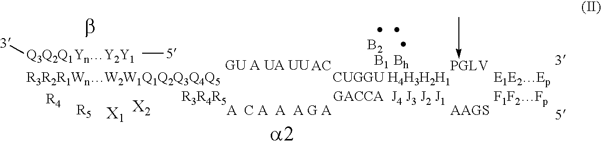
[wherein α2 represents a ribozyme sequence and β represents an oligonucleotide sequence with symbols used herein conveying the same meanings as used in general formula (I).]

6. The hairpin ribozyme according to claim 1, which is a trans-form ribozyme cleaving another ribonucleotide sequence upon activation.

7. The hairpin ribozyme according to claim 6, which exhibits a complex structure with an oligonucleotide as shown in general formula (III) 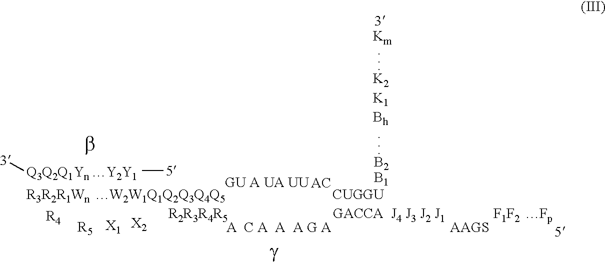
[wherein γ represents a ribozyme sequence and β represents an oligonucleotide sequence:
in which U represents a uracil nucleotide, C represents a cytosine nucleotide, A represents an adenine nucleotide, and G represents a guanine nucleotide;
B1 to Bh, F1 to Fp, J1 to J4, K1 to Km, Q1 to Q5, W1 to Wn, and X1 and X2, which may be the same or different, each represent any of a uracil nucleotide, an adenine nucleotide, a cytosine nucleotide, or a guanine nucleotide;
R1 to R5 and Y1 to Yn each represent a nucleotide, which is complementary to Q1 to Q5 and W1 to Wn, respectively;
S represents an adenine nucleotide or a cytosine nucleotide; and
h is an integer from 3 to 20, m is an integer from 1 to 10, n is an integer from 1 to 10, and p is an integer from 1 to 10.]

8. An isolated DNA encoding a ribonucleotide which constitutes the hairpin ribozyme according to claim 1.

9. A recombinant vector comprising the DNA according to claim 8.

10. A host cell into which the recombinant vector according to claim 9 has been introduced.

11. A method for activating a hairpin ribozyme, comprising changing a stem-and-loop three-dimensional structure by hybridization between an oligonucleotide and an inactive ribozyme.

12. The method for activating a hairpin ribozyme according to claim 11, wherein one or more nucleotides in the oligonucleotide are 2′-O-methylated.

13. A method for detecting a target nucleotide sequence with the hairpin ribozyme according to claim 1.

14. The method for detecting according to claim 13, comprising detecting the presence of a target nucleotide sequence in a sample contained on a DNA chip.

15. A method for detecting a target nucleotide sequence, comprising detecting a fragment cleaved by the self-cleavage of the hairpin ribozyme according to claim 1.

16. The method for detecting according to claim 13, comprising detecting a cleaved fragment using a fluorochrome or a radioactive label.

17. A detection kit for a target nucleotide sequence in a sample, comprising the hairpin ribozyme according to claim 1.

18. A method for cleaving a ribonucleotide sequence, using the hairpin ribozyme according to claim 1.

19. The method for cleaving according to claim 18, wherein administration of the hairpin ribozyme according to claim 1 is carried out separately from that of the oligonucleotide.

20. The method for cleaving according to claim 18, wherein one or more nucleotides in the oligonucleotide are 2′-O-methylated.

21. A pharmaceutical composition comprising the hairpin ribozyme according to claim 1.


SEQUENCE LISTING (7 sequences)

SEQ ID NO: 1 - DNA, 122 bp, Artificial Sequence
  Description of Artificial SequenceSynthetic DNA
cggcgaattctaatacgactcactatagggaaacagagaagtcaaccagagaaacaatctgcatctcactcgagatgtatattacctggtacccccctgacagtcctgtttggatccagctc

SEQ ID NO: 2 - RNA, 84 nt, Artificial Sequence
  Description of Artificial SequenceSynthetic RNA
gggaaacagagaagucaaccagagaaacaaucugcaucucacucgagauguauauuaccugguaccccccugacaguccuguuu

SEQ ID NO: 3 - DNA, 121 bp, Artificial Sequence
  Description of Artificial SequenceSynthetic DNA
cggcgaattctaatacgactcactatagggaaacagagaagtcaaccagagaaacaatcgcatctcactcgagatgtatattacctggtacccccctgacagtcctgtttggatccagctc

SEQ ID NO: 4 - RNA, 83 nt, Artificial Sequence
  Description of Artificial SequenceSynthetic RNA
gggaaacagagaagucaaccagagaaacaaucgcaucucacucgagauguauauuaccugguaccccccugacaguccuguuu

SEQ ID NO: 5 - DNA, 53 bp, Artificial Sequence
  Description of Artificial SequenceSynthetic DNA
cggcgaattctaatacgactcactatagggaaacagagaagtcaaccagagaa

SEQ ID NO: 6 - DNA, 89 bp, Artificial Sequence
  Description of Artificial SequenceSynthetic DNA
gagctggatccaaacaggactgtcaggggggtaccaggtaatatacatctcgagtgagatgcagattgtttctctggttgacttctctg

SEQ ID NO: 7 - DNA, 88 bp, Artificial Sequence
  Description of Artificial SequenceSynthetic DNA
gagctggatccaaacaggactgtcaggggggtaccaggtaatatacatctcgagtgagatgcgattgtttctctggttgacttctctg